(19) United States
(12) Patent Application Publication
(10) Pub. No.: US 2005/0034990 A1
(43) Pub. Date: Feb. 17, 2005

(54) System and method for electrokinetic trapping and concentration enrichment of analytes in a microfluidic channel

(76) Inventors: Richard M. Crooks, College Station, TX (US); Takashi Ito, Manhattan, KS (US); Li Sun, College Station, TX (US); Jinhau Dai, Lansing, MI (US); Rahul R. Dhopeshwarkar, College Station, TX (US)

Correspondence Address: BAKER BOTTS L.L.P., 2001 ROSS AVENUE, SUITE 600, DALLAS, TX, 75201-2980, US

(21) Appl. No.: 10/916,320

(22) Filed: Aug. 11, 2004

Related U.S. Application Data
(60) Provisional application No. 60/494,399, filed on Aug. 12, 2003.

(51) Int. Cl. G01L 1/20; C07K 1/26

(52) U.S. Cl. 204/450; 204/600

(57) ABSTRACT

According to one embodiment of the invention, a method for chemical analysis includes providing a device having a drain region, a source region, and a gate region disposed therebetween, associating a buffer solution with the drain region and the source region, causing a potential difference between the drain region and the source region until a stable current is reached, replacing the buffer solution in the source region with a solution containing an analyte, and applying a negative potential to the source region to create a forward bias.

### RELATED APPLICATIONS

This application claims the benefit of Ser. No. 60/494,399, entitled “Electro kinetic Trapping and Concentration Enrichment of Analytes in a Microfluidic Channel,” filed provisionally on Aug. 12, 2003.

### GOVERNMENT RIGHTS

This invention was made with Government support from the Department of Energy, Basic Energy Sciences, Contract No. DE-PG03-01HER15247. The Government may have certain rights in this invention.

### TECHNICAL FIELD OF THE INVENTION

The present invention relates generally to the field of microfluidics-based analysis and, more particularly, to system and method for electrokinetic trapping and concentration enrichment of analytes in a microfluidic channel.

### BACKGROUND OF THE INVENTION

Sample enrichment or preconcentration plays an important role in chemical separation and analysis. In general, if molecules to be detected (the analyte) exist in a chemical sample in high concentrations, then it is easier to detect their presence. However, many important analytes, especially biomoleculesa such as DNA, proteins, antibodies, antigens, and polysaccharides, often exist in minute quantities in real, unprocessed chemical samples.

Microfluidic devices typically consist of a network of channels, which have cross-sectional dimensions of tens to hundreds of microns, terminated with reservoirs that contain analytes. Various approaches have been used to move analytes out of the reservoirs and into the channel network, where the analytes are separated and detected. Because the total fluid volume of a microfluidic device is very small, the analyte must exist in a sufficiently high concentration for there to be enough molecules present to be detectable.

Some methods are based on controlling the electrokinetic properties of at least two plugs (zones of solution of limited or defined length within a microchannel) of an electrolyte solution. These methods, which include field-amplification stacking, isotachophoresis, and micelle sweeping, require that neighboring electrolyte plugs have different compositions. However, this is a difficult condition to realize in a commercially viable system. Other methods are based on the principle of size exclusion, which is essentially a filtration method. In this case, the analyte is larger than the pores of a filtration membrane barrier and, as such, the analyte is retained by the filter while small molecules (solvent and electrolyte) pass through.

### SUMMARY OF THE INVENTION

According to one embodiment of the invention, a method for chemical analysis includes providing a device having a drain region, a source region, and a gate region disposed therebetween, associating a buffer solution with the drain region and the source region, causing a potential difference between the drain region and the source region until a stable current is reached, replacing the buffer solution in the source region with a solution containing an analyte, and applying a negative potential to the source region to create a forward bias.

Embodiments of the invention provide a number of technical advantages. Embodiments of the invention may include all, some, or none of these advantages. Some embodiments of the invention provide devices and methods for the manipulation of charged analytes, such as molecules, particles, beads, and the like, such that the charged analytes may be trapped, filtered, fractionated, or locally enriched in concentration. Having been so manipulated, the charged analytes may be available for detection, reaction, collection, or other processing, within the same device or upon removal to another device or instrument. The portion of the device responsible for trapping, filtering, fractionating or enriching the concentration of the charged analyte may be the sole functional aspect of the device, although generally this portion will be just one component of an integrated device, i.e., a microfluidic device that is capable of performing other operations in combination with, either preceding or following, the operation disclosed herein as the subject invention. Examples of other operations performed within microfluidic devices include mixing, metering, binding, incubating, thermocycling, reacting, electrophoresing, absorbing or adsorbing and desorbing, extracting, etc., employing structures such as valves, channel networks, pressure actuators, thermal sources and sinks, and pH, conductivity, temperature or pressure sensors, which are known by and familiar to those skilled in the art.

Some embodiments may provide a method for concentrating charged analytes prior to further processing within an integrated microfluidic device. Accordingly, one embodiment provides an improved method of electrophoretic separation analysis of charged analytes based on concentrating analytes as herein described and then releasing the concentrated species into a separation channel. Alternatively, further processing may involve releasing the concentrated analytes from a first gate region and passing the concentrated species via a fluidic network to another, second gate region wherein the species may be again concentrated, collected, detected or fractionated.

Some embodiments may provide a linear series of gate regions in combination in a manner such that each gate region acts to concentrate a subset of the charged analytes adjacent thereto, while another subset passes through the gate channels of that gate region and towards the next, wherein each successive gate region presents a different threshold for passage based on electrophoretic mobility and thus causing a different subset of charged analytes to concentrate adjacent thereto.

Various embodiments of the invention may find general application in microfluidic devices for a variety of purposes because the method relies upon the balancing of physical forces and motions. As such, the invention does not require, for example, molecule-specific binding interactions in order to provide trapping, filtering, fractionation or enrichment of the analyte. Thus, the devices and methods are broadly applicable to a wide range of analytes that are charged, or may be associated with charge-bearing species, such as molecules or particles.

Other technical advantages are readily apparent to one skilled in the art from the following figures, descriptions, and claims.

### BRIEF DESCRIPTION OF THE DRAWINGS

For a more complete understanding of the invention, and for further features and advantages, reference is now made to the following description, taken in conjunction with the accompanying drawings, in which:

FIG. 1 is a schematic of a system that illustrates a principle of electrokinetic trapping and concentration enrichment of analytes in a microfluidic channel according to an embodiment of the invention;

FIG. 2 illustrates a system for electrokinetic trapping and concentration enrichment of analytes in microfluidic systems according to one embodiment of the invention;

FIGS. 3A through 3C are fluorescence micrographs of a fluidic device used to demonstrate one embodiment of the invention;

FIG. 4 is a graph illustrating fluorescence intensity as a function of time allotted for concentration enrichment of DNA in an experiment used to demonstrate one embodiment of the invention;

FIG. 5 illustrates a system for electrokinetic trapping and concentration enrichment of analytes in microfluidic systems according to one embodiment of the invention in which an enriched band of a mixture may be utilized as an injection plug for electrokinetic separation;

FIGS. 6A and 6B illustrate systems for electrokinetic trapping and concentration enrichment of analytes in microfluidic systems according to embodiments of the invention in which the location of an enriched band may be controlled by applying a sequence of bias voltages with well-defined temporal control; and

FIG. 7 illustrates a system for electrokinetic trapping and concentration enrichment of analytes in microfluidic systems according to one embodiment of the invention in which a mixture of charged molecules or objects may be trapped, enriched, and separated using a series of gate channels.

### DETAILED DESCRIPTION OF THE INVENTION

Embodiments of the present invention provide methods and/or devices for the trapping and enrichment of charged analytes and, as described herein, is based on a fundamentally new principle that is broadly applicable to a wide range of uses. However, the present invention contemplates that the principle is general and versatile and should be applicable to any suitably charged molecule or object, and to many other suitable forms of a device in which the principle operates.

In the following detailed description, the term “analyte” is used in a broad sense. On one hand, analyte means a discrete substance, molecule, aggregate, polymer, bead, particle, cell or subcellular component, bearing a charge, that is to be subjected to manipulation or processing in a device or by a method. Analytes include, but are not limited to, peptides, proteins, polynucleotides, polypeptides, oligonucleotides, organic molecules, haptens, epitopes, cells or parts of biological cells, posttranslational modifications of proteins, receptors, complex sugars, vitamins, hormones, and the like. Analytes may also be inorganic compounds or particles, semiconductor particles, such as quantum dots, polymeric beads or particles, etc., provided the material in question is small enough to be able to physically pass through gate channels of a particular device (as described in greater detail below) and thus be capable of performing according to inventive methods described herein. For any of the above-listed exemplary analytes, they must either have a net anionic or cationic charge; though if not intrinsically charged, the particular analyte may be modified or otherwise associated with some other charged species such that it bears a charge when used in embodiments of the invention. However, the term “analyte” is not intended to refer to the ions that make up the supporting electrolyte used in the buffer solutions.

On the other hand, and according to the context, “analyte” is also used to mean a multicomponent sample used according to embodiments of the invention. Used in this sense, the analyte contains a plurality of distinct charged species, and each of the species will respond differently in the operation of a device. Some of the species may become concentrated, while others may not under a given set of conditions.

FIG. 1 illustrates a system 100 for electrokinetic trapping and concentration enrichment of an analyte 111 according to one embodiment of the invention. FIG. 1 is meant to illustrate the general principle of the invention, which relies on exerting spatial control over the electrokinetic velocity of analyte 111. In the illustrated embodiment, system 100 includes a drain region or channel 102 having a first electrode 103, a source region or channel 104 having a second electrode 105, a gate region 106 providing fluid communication between drain channel 102 and source channel 104, a buffer solution 108 associated with drain channel 102, a second solution 110 containing analyte 111 disposed in source channel 104, and a power supply 112 operable to impart a potential difference, or bias, between first electrode 103 and second electrode 105.

In the illustrated embodiment, gate region 106 is defined by a plurality of gate channels 107 that span between drain channel 102 and source channel 104. The gate channel openings are pores that are filled with electrolyte buffer solution 108. The size of any one gate channel 107 (or pore opening) should be smaller than the cross-sectional area of the channel section that gate channel 107 opens into (i.e., the area of the gate channel/channel section interface) in order to have a functional device. In one embodiment, the combined area of the openings of all gate channels 107 is prescribed to be smaller than the cross-sectional area of the interface between gate region 106 and the drain or source channels, as further described below. The area of the interface is in some embodiments equivalent to the cross-section of a channel section as is generally the case when the channel sections lie within the same plane.

On the other hand, the present invention also calls for the pore openings to be larger than the size of the analyte that is to be trapped, filtered, or concentrated using the device. As such, pore openings, or gate channel widths, ranging in scale from about 2 nm to 2 μm, or even 5 μm are contemplated. A range of 10 nm to 1000 nm may be more typical. Accordingly, for a device with channel sizes on the order of 10 μm to 1 mm, the relative width of the pore opening to the width of the channel sections is at least a ratio of about 1 to 10, and may be as small as 1 to 1,000 or 1 to 100,000. The size of opening chosen depends on a variety of factors, such as the size, the charge and mobility of the analyte, the viscosity and conductivity of the buffer solution, the chemical nature of the gate region material, and the anticipated rate of convective flow required through the pores.

The latter consideration, the rate of convective flow, is a significant factor in the operation of a device. Electroosmotic (eo) flow is generated within a gate channel 107, as indicated by arrow 109, when an electric field exists along that channel as a result of there being a permanent charge on the wall of the channel. Counterions predominate in the solution double layer at the wall interface, and the electrokinetic movement of these counterions in response to the electric field causes a net flow of the bulk solution. In one operation of the device, convective flow is required through gate channels 107 and, as such, channel walls bearing a charge are a characteristic of the device. At least one of either drain channel 102, source channel 104 or gate channels 107 of gate region 106 will have a charge-bearing surface. With an eo flow generated in at least one of these sections there will be a net convective flow (a combination of eo flow and pressure-driven flow caused by the eo flow) of buffer solution 108 in gate channels 107, due to the requirements of mass balance, subject to considerations such as the particular geometry of the particular channel network in a device.

Electrodes 103, 105 are said to be associated with their respective channel sections 102, 104. By this it is intended that a particular electrode sets the electrical potential distribution in the solution that is contained in its respective channel section, proximal to gate region 106. The electrode may reside within the channel, or within a suitable port or reservoir that communicates with the channel. In the case of a device being comprised of a network of channels, the electrode may be physically distant, and may even be located on the other side of a different gate region. However, for the gate region in question, the electrode is considered associated with a channel section as long as the channel section that the electrode sets the potential of is proximal with respect to the gate.

With reference to FIG. 1, an operation of one embodiment of system 100 as it embodies a method for concentrating a charged analyte is now considered. Drain channel 102 and source channel 104 of the microfluidic device are filled with electrolyte buffer solution 108. The electrolyte provides ion conductors within the fluid and in response to a potential bias supplied between electrodes 103, 105 supports the establishment of an electric field along the channels. Analyte 111, specifically a negatively charged analyte, such as a DNA molecule, is initially located in source channel 104, typically in a port or reservoir that communicates with the channel section, thereby associating it with the channel section. A potential bias is imparted to electrodes 103, 105 to produce an electric field therebetween, wherein in this example the source electrode 105 is negative and drain electrode 103 is positive.

Accordingly, the electrokinetic motion of the DNA molecule under the influence of the electric field is towards drain electrode 103, and thus towards the gate region 106. This motion is characterized by an electrokinetic velocity that is the vector sum of the intrinsic electrophoretic (ep) velocity of analyte 111 and the convective velocity of buffer solution 108. Again, convective motion of buffer solution 108 refers to motion that is either electroosmotic flow or pressure-driven flow induced by the eo flow, or a combination thereof. By providing wall sections of any or all of the channels that bear a negative charge, the convective velocity of buffer solution 108 within gate region 106 is opposite in direction to the ep velocity of DNA in source channel 104. By having a convective velocity that is larger than the ep velocity, the DNA is not able to enter gate region 106 and instead accumulates and concentrates at a location in source channel 104 adjacent gate region 106. The location of the concentrated band that forms is typically near the interface of gate region 106 with source channel 104.

As shown in FIG. 1, the first requirement for trapping and enriching DNA (i.e., analyte 111) is that the ep velocity of DNA in source channel 104, which may be polydimethylsiloxane (PDMS), must be larger than the eo velocity of buffer solution 108 in source channel 104. That is to say, analyte 111 has a net electrokinetic velocity in source channel 104 towards gate region 106. The second requirement for DNA trapping and enrichment is that the local convective velocity of buffer solution 108 in the gate channels 107 is greater than and opposite in direction to the intrinsic ep velocity of analyte 111. Thus, fluid emanating from the pores, or gate channels 107, exemplified by, for example, a polyethylene terephthalate (PETE) membrane, effectively prevents analytes 111 from entering. Accordingly, analyte 111 accumulate near gate region 106 due to the opposing forces.

Because the eo velocity is equal to the product of the eo mobility and the electric field, thus it follows that the eo velocity may be modulated by changing the magnitude of the electric field. Furthermore, the local eo velocity may be varied according to position within a microfluidic network by simply changing the cross-sectional area at that position. For example, having a gate region comprised of gate channels having a smaller total cross-sectional area compared to the interface of the gate region and the source channel results in a higher local eo velocity through the pores. Thus, changing the cross-sectional area ratio provides a means for modulating the eo (convective) flow within a channel. This flow rate modulation selectively traps those analytes having an intrinsic ep mobility smaller than the local velocity of the eo “jets” emanating from the gate channel, while passing those analytes that have a ep mobility higher than the eo jet velocity.

Referring to FIG. 1, in the case of analyte 111 being positively charged, the particular parameters given in the above example would need to be changed appropriately. For example, the bias imparted would be reversed, with source electrode 105 being positive and drain electrode 103 being negative, and the walls would necessarily have a net positive charge in order that an opposing convective flow be established through gate channels 107.

Gate region 106 and gate channels 107 associated therewith are an important part of the subject invention. In one embodiment, the use of a porous membrane, more usually referred to as a nanoporous membrane, is contemplated. Nanoporous membrane materials are an area of interest, particularly for applications in filtration and sensing. As a result, methods for controlling the pore size, the composition, and the functional groups exposed within the pores, and the use of materials that range from organic polymers to semiconductors, ceramics and organic/inorganic composites, such as epoxy-embedded carbon nanotubes are known in the art. Depending on the material used, those skilled in the art will be familiar with the various techniques used to prepare such membranes, such as ion-track or ion-beam etching, anodic etching, microlithography in combination with etching, laser ablation, templated chemical assembly, sol-gel techniques, and the like. Nanoporous membranes for use in some embodiments of the invention may be comprised of at least one of the following: a polyester polymer such as PETE, polyimide, cellulose, polycarbonate, carbon or carbon nanotubes, semiconductors such as silicon or insulators such as silicon nitride, silica, alumina, or other inorganic ceramic materials such as titanates. Another class of nanoporous material, the hydrogel, is also useful in the invention and is described in further detail below.

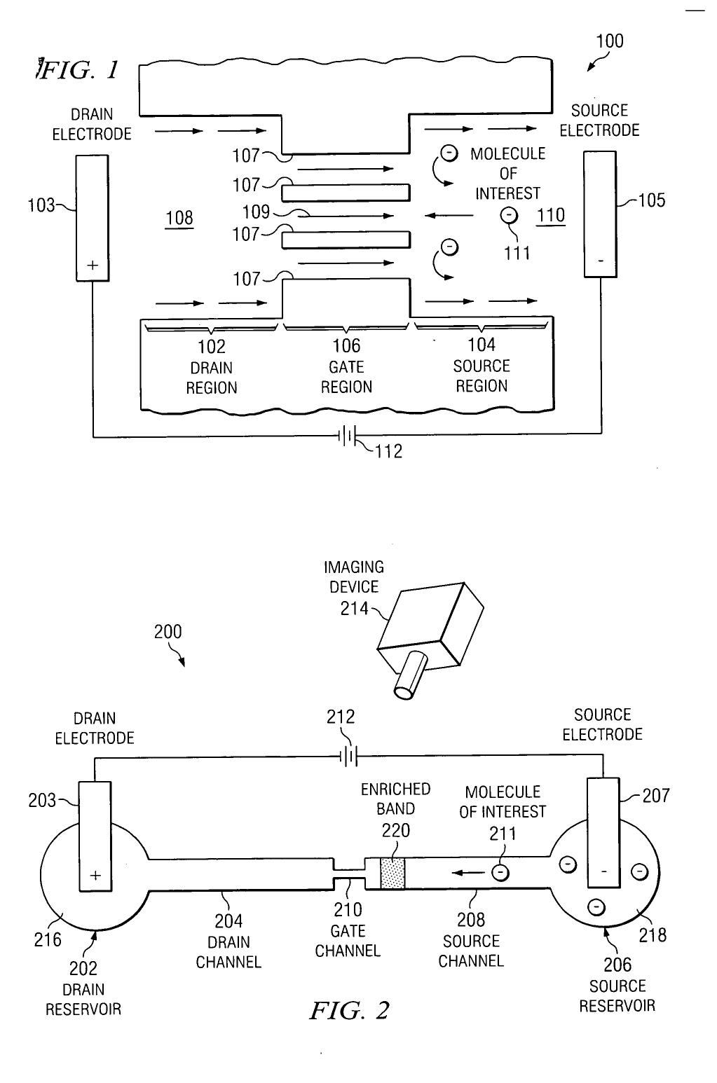

FIG. 2 illustrates a microfluidic system 200 chemical analysis and separation in accordance with one embodiment of the invention. In the illustrated embodiment, system 200 includes a drain reservoir 202 having a first electrode 203, a drain channel 204, a source reservoir 206 having a second electrode 207, a source channel 208, gate channel 210, a power supply 212, and an imaging device 214. Drain reservoir 202 may be any suitable size and shape and contains a buffer solution 216. Drain reservoir 202 couples to drain channel 204 in any suitable manner. Drain channel 204 may be any suitable size and shape; however, in one embodiment drain channel 204 as well as source channel 208 are polydimethylsiloxane (PDMS) channels having an approximate cross-section of 100 μm×20 μm. Source reservoir 206 also may be any suitable size and shape and contains a second solution 218 containing one or more analytes 211. Gate channel 210 may be any suitable size and shape; however, in the illustrated embodiment, gate channel 210 is a nanoporous polyester membrane. For example, in a particular embodiment of the invention, gate channel 210 is a polyester membrane formed from PETE and having a 200 nm pore diameter, 10 μm thick, and 3×10⁸ pores/cm² manufactured by Osmonics. In other embodiments, gate channel 210 may be a porous hydrogel polymer network.

First electrode 203 and second electrode 207 may be any suitable electrodes and are immersed within their respective reservoir in order to create an electric field inside drain channel 204 and source channel 208. Power supply 212, which may be any suitable power supply, is operable to apply a potential difference between first electrode 203 and second electrode 207.

Imaging device 214 may be any suitable device that is operable to create an image of analyte 211 within system 200. For example, imaging device 214 may be an inverted fluorescence microscope equipped with an imaging CCD camera. In one embodiment, imaging device 214 is operable to obtain fluorescence micrographs of analyte 211.

In operation of one embodiment of system 200, drain reservoir 202, drain channel 204, source reservoir 206, and source channel 208 are filled with buffer solution 216. Buffer solution 216 may be any suitable buffer solution and, in a particular embodiment, buffer solution 216 is a TBE buffer solution (89 mM TRIS base+89 mM boric acid+2 mM EDTA, pH 8.4) at reduced pressure. Buffer solution 216 is conditioned by applying 100 V between first electrode 203 and second electrode 207 until a stable current is reached. That portion of buffer solution 216 in source reservoir 206 is then replaced with second solution 218 containing analyte 211. For example, in a particular embodiment of the invention, analyte 211 is 10 μg/mL DNA (a 20 mer ssDNA, 5′-labeled with fluorescein, obtained from IDT of Coralville, Iowa.). Although analyte 211 is labeled with fluorescein in this example, any suitable label may be used to label analyte 211.

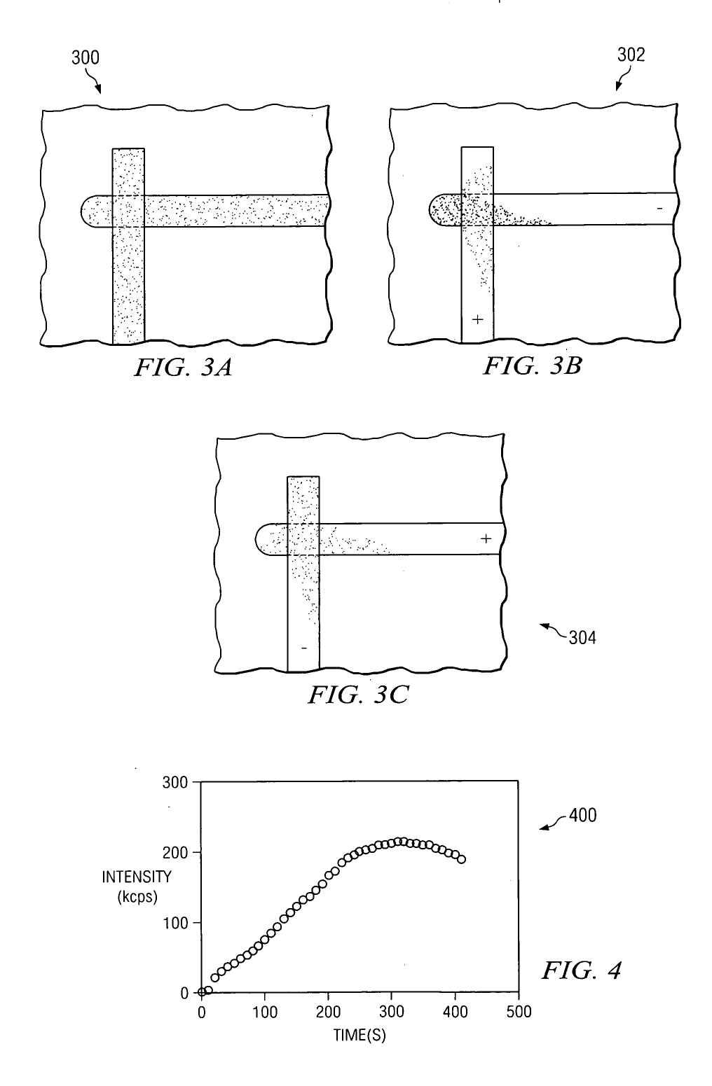

After obtaining a fluorescence micrograph 300 (FIG. 3A), a forward bias (negative potential in source reservoir 206) is applied between first electrode 203 and second electrode 207. The resulting motion of analyte 211 (i.e., DNA) is recorded using imaging device 214. This eventually results in the formation of an enriched band 220 of analyte 211 within source channel 208. Using the conditions noted above, enrichment of DNA is apparent within approximately thirty seconds and reaches an enrichment factor of eleven within approximately sixty-eight seconds, as shown in the fluorescence micrograph 302 of FIG. 3B. The minimum width of enriched band 220 is approximately 100 μm. The formation of enriched band 220 is a result of the general principle of the invention, as outlined and described in FIG. 1.

When the forward bias is reversed, analyte 211 is immediately transported through gate channel 210, which indicates that enrichment is not a consequence of size exclusion induced by gate channel 210 (in this example the pores of the nanoporous membrane). Transportation of analyte 211 through gate channel 210 is illustrated by the fluorescence micrograph 304 of FIG. 3C. The transported analyte 211 is then trapped in drain channel 204 by the same balance of ep and eo velocities that was initially responsible for the formation of enrichment band 220 in source channel 208.

FIG. 4 is a graph 400 illustrating fluorescence intensity as a function of time allotted for concentration enrichment of DNA in an experiment used to demonstrate one embodiment of the invention. As illustrated in FIG. 4, the magnitude of DNA concentration reaches a limiting value in the center of the enriched band within a finite time (t_{conc}). At t<t_{conc} the enriched band has a nearly constant length, but when t>t_{conc} this enriched band begins to expand longitudinally in the direction of the source reservoir. Using the conditions noted above, for an initial concentration of 10 μg/mL DNA and a 100 V forward bias, tconc is approximately five minutes and the enrichment factor, calculated from the relative fluorescence intensity is approximately thirty. However, when the DNA concentration in the source reservoir is reduced to approximately 1 μg/mL and 0.1 μg/mL, the enrichment factor is increased to 300 and 800, respectively. Considering the simplicity and compactness of a microfluidic system, such as system 200, these enrichment factors are significant.

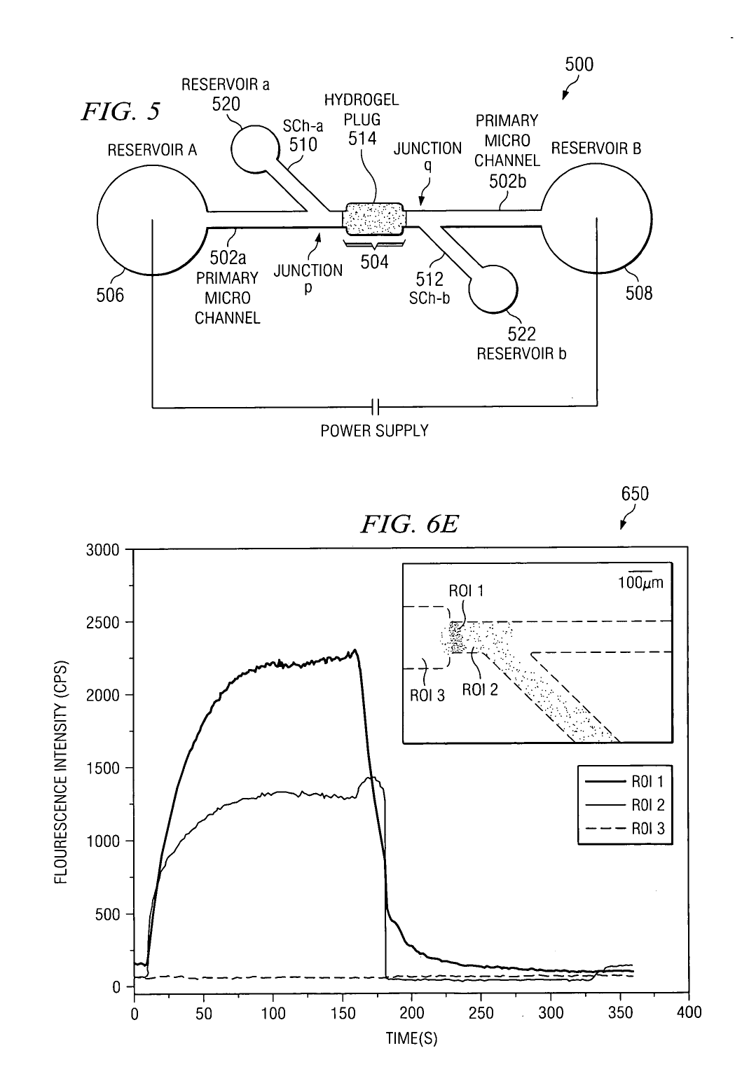

FIG. 5 illustrates a system 500 for implementing another embodiment of trapping and enrichment of a negatively charged molecule or object according to the teachings of the invention. In the illustrated embodiment, system 500 includes a microfluidic device having a primary microchannel 502 that has a section 504 with an enlarged width at the center, and a first reservoir 506 and a second reservoir 508 at either primary microchannel 502 terminus. Side channels 510, 512 are also shown intersecting with first and second channel sections 502a, 502b of the primary channel 502, although these are optional and not critical to the concentrating function of the device. The enlarged width section 504 is used to hold a hydrogel plug 514 that serves as the gate region of the device.

A PDMS-glass microfluidic device similar to system 500 was fabricated for testing using standard rapid prototyping procedures and techniques. The microfluidic channel network formed had primary microchannel 502 about 7 mm long whose ends were connected to reservoirs 506, 508, each 3 mm in diameter. This primary microchannel 502 was approximately 100 μm wide and 25 μm deep, except at center section 504 where it was approximately 200 μm wide for a length of about 400 μm. On either side of section 504, that was to be the gate region, there was side channels 510, 512 connected to primary microchannel 502 at arbitrary angles (˜450°). The side channels 510, 512 terminate in reservoirs 520, 522, each about 0.5 mm in diameter. The cross-sectional dimensions of side channels 510, 512 were the same as those for primary microchannel 502.

The gate region hydrogel plug 514 was prepared in situ by first placing a hydrogel precursor solution comprising 1:4 molar ratio of acrylic acid (AA) to 2-hydroxyethyl methacrylate (HEMA), 5 wt % of a crosslinker, ethylene glycol dimethacrylate (EGDMA) and 3 wt % of a photoinitiator, 2,2-dimethoxy-2-phenyl acetophenone (DMPAP) (all from Sigma-Aldrich, Inc. St. Louis, Mo.), into primary microchannel 502 by capillary action. A UV beam of 365 nm (300 mW/cm², EFOS Lite E3000, Ontario, Canada) was projected onto the gate region for about 200 s from the side port of a microscope (DIAPHOT 300, Nikon) and through a 10× objective. A chrome mask was placed at the confocal plane at the side port so that a well-defined pattern of UV beam was created prior to projection and reduction through the microscope optics. Unreacted precursor solution was flushed out by introducing 10 mM Tris-HCl buffer solution (pH=8.3) at a rate of 10 μL/min through reservoirs 506, 508 for more than 10 min. As the anionic hydrogel plug 514 comes in contact with the basic buffer solution, it expands and pushes itself against the walls of microchannel 502. Comparatively less swelling is observed in case of uncharged hydrogel plug; still, it is enough to ensure that plug 514 remains stationary even under the influence of electric field.

The enlarged width section 504 in the primary channel 502 is advantageous for securing the position of hydrogel plug 514, and the prevention of any movement or slippage of the plug along the channel is desired. It is preferable to stabilize hydrogel plug 514 against the influence of high electric fields or other forces, such as hydrodynamic flow either during device fabrication or usage of the device. Alternatively, or in addition to the flange provided by enlarged width section 504, the channel walls may be treated to have a reactive chemical group (e.g., a crosslinking agent) that can chemically bond to hydrogel plug 514 in order to secure its position.

Suitable hydrogel polymer plugs may be prepared from a wide variety of monomers, crosslinking agents, and initiators. The various components may be chosen for their degree of hydrophilicity, net charge, ability to enhance or reduce swelling or specific functional groups they may possess. However, the gel should provide two basic properties: (1) an ability to act as a porous ion conductor, and (2) the structural strength to withstand the forces of the electric field and any pressure-driven flow to which it will be exposed. In this regard, the amount of crosslinker used is important. When the percentage amount is too low the gel will not be rigid enough. Conversely, too high an amount of crosslinker limits the ability to swell and form a porous network. Thus, a percentage of at least about 5%, and no more than about 10% is preferred. Likewise, the amount of initiator used determines the properties of the gel produced. Initiator present in the range of ˜1-5 wt % generally produces hydrogels suitable for the present invention.

The hydrogel plug 514 may be preformed and added to the microfluidic channel network, or it may be formed in situ. The formation of the polymer may be photoinitiated or thermally initiated. Photoirradiated regions may be determined by interposing a mask or using a directed light source. On the other hand, the location of the gel precursor solution may be defined and thus determine the location and size of hydrogel plug 514. These methods are also well known in the art. Another exemplary method has been reported by Yu, et al., in Anal. Chem., 2002, 73, p. 5088-5096.

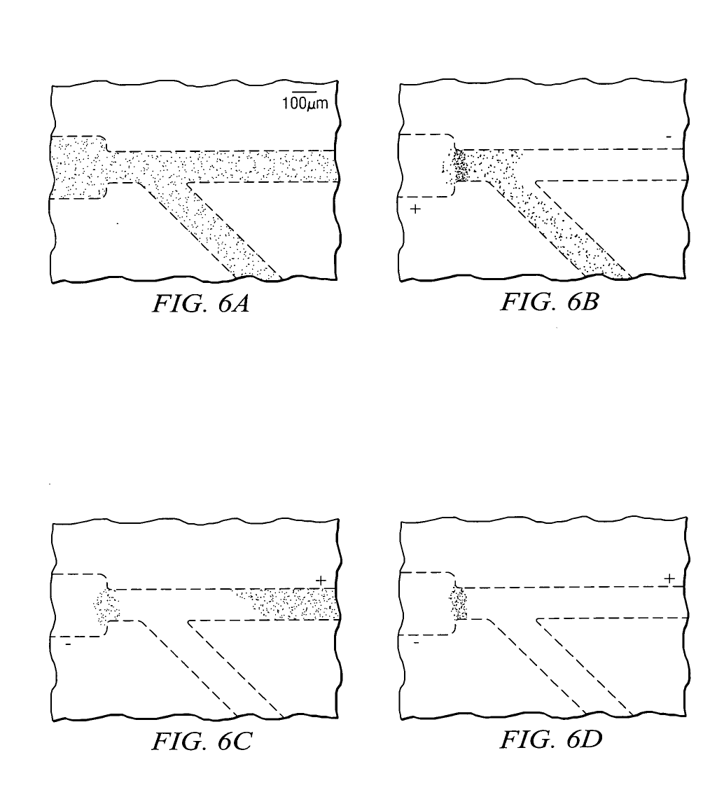

FIGS. 6A through 6D illustrate the preconcentration phenomena observed in a microfluidic channel incorporating the anionic hydrogel plug 514, illustrated by system 500, as imaged using the microscope system previously described. After conditioning the channel by applying a potential bias of 100 V for 10 min, the potential was switched off and a 5 μM fluorescein solution was introduced through reservoir 508 to replace the buffer solution in channels 502b and 512. The solutions in all the reservoirs 506, 520, 508, 522 are kept at the same level to nullify any hydrodynamic flows inside the channel. Two platinum electrodes were inserted in reservoirs 506, 508. A potential of 100 V was applied between the electrodes using a power source (range 0-1067 V) operated by a suitable in-house computer program.

FIG. 6A shows the fluorescence micrograph obtained before the application of any potential bias. After applying a forward 100 V bias (reservoir 506 at positive potential), the negatively charged fluorescein ions migrate rapidly from reservoir 508 to reservoir 506. However, hydrogel plug 514 at the gate region acts as a barrier to this movement, resulting in the concentration of fluorescein near the hydrogel-solution interface. This is apparent from FIG. 6B, where concentration factors of 16 and 10 were achieved just inside the hydrogel and in the solution just outside the hydrogel, respectively, within 90 s. During forward bias, some fluorescein was lost as a fraction of fluorescein solution (about 2-fold concentrated) is directed towards floating reservoir 522. When the potential bias is reversed (FIG. 6C), fluorescein is rapidly transported back towards reservoir 508. A minute amount of fluorescein (about 0.75-fold) was trapped inside hydrogel plug 514 even after the application of reverse bias for 120 s (FIG. 6D).

As shown by a graph 650 of concentration vs. time in FIG. 6E, a higher preconcentration factor is observed inside hydrogel plug 514 (ROI 1) than in the solution (ROI 2). The enrichment factors reach a limiting value of 16 and 10 respectively within 100 s. At t=160 s, in the absence of potential bias, fluorescein concentrated inside hydrogel plug 514 starts to diffuse back to the solution, which in turn increases the concentration at ROI 2. The above observation is likely caused by electrostatic repulsions between the hydrogel backbone and the fluorescein molecules, which become significant when the external bias voltage is turned off. On applying a reverse bias at t=180 s, fluorescein is immediately transported back towards reservoir 508.

In a recent study of protein interaction and diffusion in HEMA-co-AA hydrogels, it has been reported that negatively charged protein Bovine Serum Albumin (Molecular weight 66 kDa and hydrodynamic radius of 3.4 nm at 4° C.) is able to diffuse (diffusion coefficient of the order of 10⁻⁸ cm²/s) through the hydrogel at swelling ratios of less than 2. This suggests that the hydrogel pore size is greater than 3.4 nm. The calculated Debye length for 10 mM buffer solution is about 3 nm. Thus, the hydrogel pore size is sufficiently larger than the Debye length, giving rise to considerable electroosmotic flow inside the pores. The observed concentration phenomenon is consistent with the explanation that an electroosmotic flow opposes the electrokinetic transport of fluorescein ions, with a balance being achieved just inside the hydrogel boundary where sample stacking, i.e., the formation of the band of fluorescein, is observed. Electrostatic repulsion between the charged hydrogel and the anionic analyte is also present, although experiments using uncharged hydrogel display the same stacking, or concentration, phenomenon.

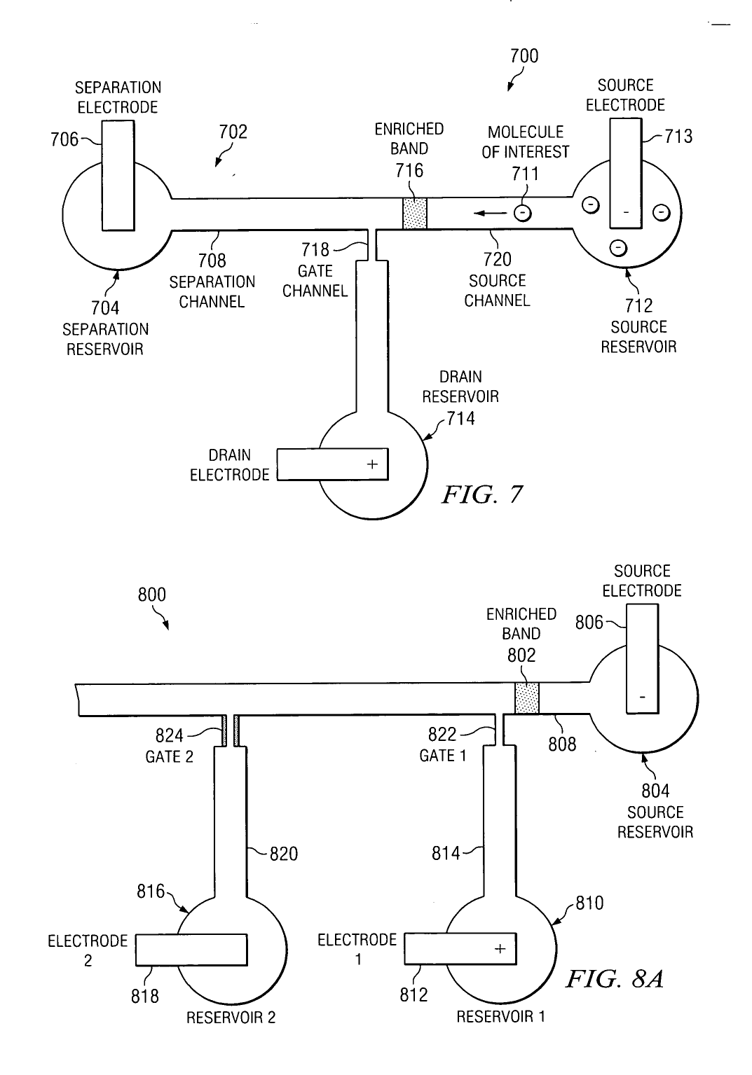

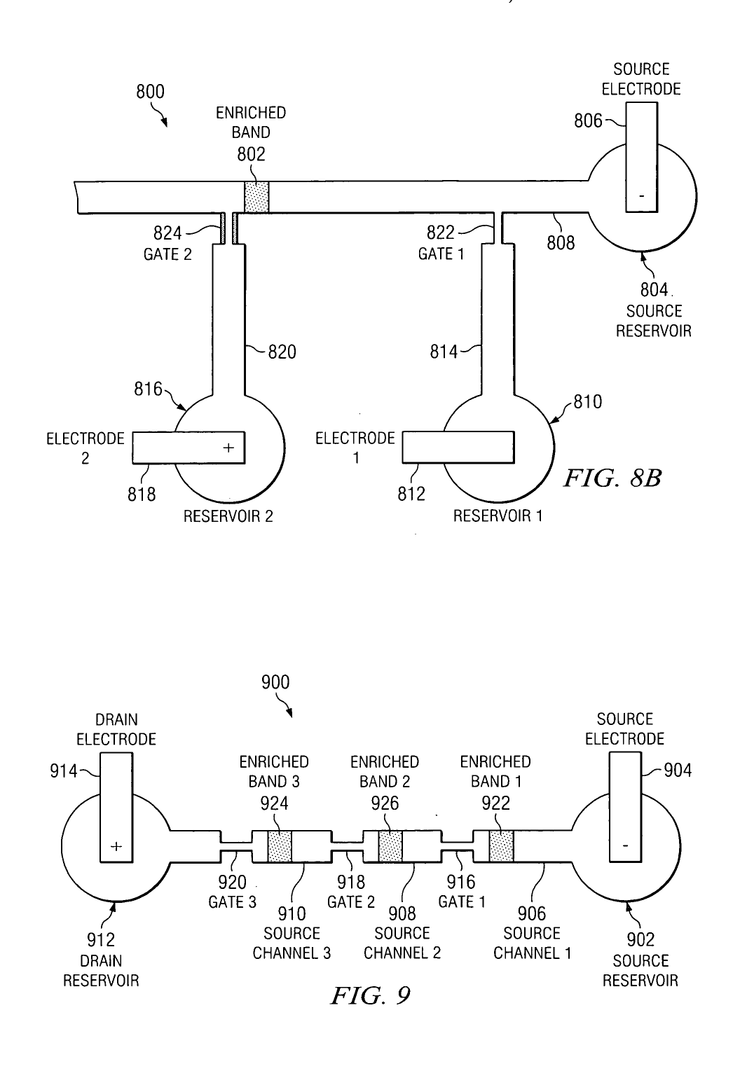

The trapping and/or enrichment principles illustrated and described above in conjunction with FIGS. 1 through 6 may be important in many applications. Some of these applications are described in the embodiments illustrated in FIGS. 7 through 9.

FIG. 7 illustrates a system 700 for electrokinetic trapping and concentration enrichment in microfluidic systems according to one embodiment of the present invention in which an enriched band of a mixture may be utilized as an injection plug for capillary electrophoretic (CE) separation. System 700 may be considered to be similar to system 200 of FIG. 2; however, in the embodiment illustrated in FIG. 7, system 700 also includes a separation region 702 having a separation reservoir 704, a separation electrode 706, and a separation channel 708, which is coupled to a source channel 710. Assuming that an analyte 711 is a negatively charged DNA molecule, a forward bias (negative potential in a source reservoir 712 and a positive potential in drain reservoir 714) is applied for a particular period of time. As a result, an enriched band 716 forms in a source channel 720 adjacent a gate channel 718, in accordance with the principle described above in conjunction with FIG. 1.

When that bias is turned off, a bias is applied between a source electrode 713 and separation electrode 706 immediately thereafter, such that enriched band 716 is driven toward separation reservoir 704. Other embodiments for channel networks and the manner in which separation channel 708 communicates with gate channel 718 are also included in the subject invention. The analyte 711 concentrated at gate channel 718 may be processed in a variety of manners, such as, for example, to create a defined injection plug, prior to being driven into separation channel 708. Methods and devices for injecting sample plugs into microfluidic electrophoretic separation channels are well known in the art; see for example U.S. Pat. Nos. 5,599,432, 5,750,015, 5,900,130, 6,007,690, 6,699,377, which are herein incorporated by reference.

The separated analytes 711 may be collected at the end of separation channel 708, further injected into a separate instrument, or a detector may be positioned along or at the end of separation channel 708 to record the passage of analytes 711 along separation channel 708. Detection may be optical (absorbance, fluorescence, shadowing), electrochemical (amperometric, potentiometric), or by other suitable methods. Note that separation channel 708 may be modified to obtain a particular benefit; for example, the channel surface may be treated to modulate and control eo flow, or the entire channel may be filled with a separation medium, such as a sieving polymeric matrix for the case of DNA separation, to improve resolution.

FIGS. 8A and 8B illustrate a system 800 for electrokinetic trapping and concentration enrichment in microfluidic systems according to an embodiment of the invention in which the location of an enriched band 802 may be controlled by applying a sequence of bias voltages with well-defined temporal control. In the illustrated embodiment, system 800 includes a source reservoir 804 having a source electrode 806, a source channel 808, a first drain reservoir 810 having a first drain electrode 812, a first drain channel 814, a second drain reservoir 816 having a second drain electrode 818, a second drain channel 820, a first gate channel 822, and a second gate channel 824.

In this embodiment of the invention, enriched band 802 forms by applying a forward bias voltage between source electrode 806, and first drain electrode 812. This forward bias is then switched off and another forward bias is immediately applied between source electrode 806 and second drain electrode 818. Enriched band 802 thus moves from a location adjacent first gate channel 822 to a location adjacent second gate channel 824, as indicated in FIG. 8B. Enriched band 802 moves to this new location via ep transport. This type of manipulation of enriched bands containing highly concentrated chemical reagents may be utilized in other suitable applications. For example, using the illustrated technique to bring two reactive reagents together to initiate a chemical reaction or to bring together assay reagents for binding, interacting, or reacting is contemplated by the present invention.

FIG. 9 illustrates a system 900 for electrokinetic trapping and concentration enrichment in microfluidic systems according to one embodiment of the invention in which a mixtures of charged molecules may be trapped, enriched, and separated using a series of gate channels. As such, system 900 includes a source reservoir 902 having a source electrode 904, a first source channel 906, a second source channel 908, a third source channel 910, a drain reservoir 912 having a drain electrode 914, a first gate channel 916, a second gate channel 918, and a third gate channel 920. Gate channels 916, 918, and 920 have successively smaller cross-sectional areas with respect to their adjacent source channel to allow trapping, enrichment, and separation of a mixture of charged chemicals or objects based on differences in their ep mobilities. For example, based on the principles discussed above in conjunction with FIG. 2, after separation a first enriched band forms in first source channel 906 adjacent first gate channel 916 and contains chemicals with the lowest average ep mobility, whereas an enriched band 924 forms within third source channel 910 adjacent third gate channel 920 and contains chemicals with the highest average ep mobility. In addition, an enriched band 926 forms in second source channel 908 adjacent second gate channel 918 and contains chemicals with an average ep mobility between that of enriched band 922 and enriched band 924. A combination of the pore size, number of pores, total area of pore openings and pore surface charge of gate channels 916, 918, and 920 may be varied to achieve the proper balance of convective flow rate per pore for each gate channel.

Device Fabrication

A microfluidic device generally has a thickness of 0.2 to 10 mm, and a length and width that vary greatly depending on the purpose, but are generally in the range of 2 to 20 cm, and 2 to 10 cm, respectively. Devices for electrophoretic separations may be longer. In some embodiments, devices fabricated wholly using thin films may be used as devices and be as thin as about 0.1 mm.

Devices according to the present invention may be fabricated in any suitable manner. A device body may be formed in plastic off of a micromachined/etched positive rendition of the channels, chambers, reservoirs and any other fluidic features. Suitable plastics include acrylics, polycarbonates, polyolefins, polystyrenes and other polymers suitable for microfluidic or electrokinetic applications. A backing is preferably made of a nonconducting film or body attached to the surface of the device body containing the channels. One suitable material for a backing is a polymethylmethacrylate (PMMA) film. The backing is preferably attached to the body by chemical, thermal, or mechanical bonding. Ultrasonic welding (an example of mechanical welding) may also be employed to fuse various parts together. Of course, the body of microfluidic devices may be produced directly by etching the intended structures in a substrate. In such instances, a cover including wells or reservoir openings is preferably placed over channels or trenches in the substrate to complete the device. Alternatively, the channel features may be formed in the cover (or film) by, e.g., embossing, the film thereafter being attached to a substrate that may feature wells. Other materials are also contemplated, such as the exemplified material poly(dimethylsiloxane) (PDMS), silicon, and glass. Further details as to device construction may be appreciated by those skilled in the art.

In some embodiments, channels have a rectangular, trapezoidal, or “D”-shaped cross-section. However, other cross-sectional geometries may be employed. Preferably, the channels have a substantially constant or uniform cross-section. It is also preferred that channels have a surface finish that does not result in irregular flow effects.

Devices may also be constructed in a multilayer fashion, wherein the channels comprising the microfluidic network of the device do not all lie within the same plane. One advantage of a multilayer design is that thin porous polymer films or monoliths may be used as the gate region, which may be incorporated into the device as a layer interposed between substrate lamella in which channels, reservoirs, etc. are included. In other words, the device may be assembled by aligning, and sealing together, a channel substrate layer, a gate region layer, and a second channel substrate layer. U.S. Pat. No. 6,623,860 to Hu describes fabrication methods and designs for multilevel flow structures useful for microfluidic operations.

Material is generally added to or removed from devices at ports or reservoirs. A reservoir is preferably sized to contain sufficient material for performing a desired test or experiment and also may be used for insertion of an electrode so that an external electrode may be submerged into any fluid solution used in the device and used to apply an electric field within portions of the solution, e.g., for applications requiring electrokinetic motion (i.e., employing either or both electrophoretic and electroosmotic phenomena). Suitable materials for the electrodes include platinum or other suitable conducting materials, particularly those resistant to corrosion. The electrodes may be connected to a programmable voltage controller for applying desired voltage differentials across the channels. Alternatively, electrodes may be integrated by directly fabricating electrodes on the surface of the device. See, for example, the disclosure of Zhao et al. in U.S. Patent Application No. 20020079219, for a discussion of forming electrodes on plastic microfluidic devices. Methods for forming electrodes from metals or conducting inks on glass or polyimide substrates are also well known in the art.

As is known in the art, voltages may be used to drive the device. For example, U.S. Pat. No. 6,010,607 to Ramsey describes information on the manner in which voltages may be applied in order to operate the device. Although the present invention relies upon electrokinetic phenomena, any device using the invention need not rely exclusively on electrokinetic motivation throughout the device. As is apparent to one with skill in the art, features as described herein may be used in connection other components or modules within the microfluidic device that is at least partially pressure driven or otherwise motivated.

Although embodiments of the invention and their advantages are described in detail, a person skilled in the art could make various alterations, additions, and omissions without departing from the spirit and scope of the present invention.

1. A system for manipulating charged analytes, comprising:
a drain region having a first electrode associated therewith;
a source region having a second electrode associated therewith;
a gate region disposed between the drain region and the source region;
a first solution disposed in the drain region;
a second solution containing an analyte disposed in the source region;
the electrodes operable to produce a potential difference across the gate region; and
charge-bearing walls in at least one of the drain region, the source region, and the gate region such that convective flow occurs through the gate region when a potential is applied across the first and second electrodes.

2. The system of claim 1, further comprising an imaging device operable to detect motion of the analyte.

3. The system of claim 1, wherein the analyte is labeled with a label.

4. The system of claim 1, wherein the drain and source regions lie in the same plane.

5. The system of claim 4, wherein the drain and source regions are co-linear.

6. The system of claim 4, wherein the drain and source regions are perpendicular.

7. The system of claim 1, wherein the drain and source regions lie in different planes.

8. The system of claim 7, wherein the gate region comprises a nanoporous membrane and lies in a plane between the different planes of the drain and source regions.

9. The system of claim 1, wherein a width of the gate region is about 10-1000 nm.

10. The system of claim 1, wherein the gate region comprises a nanoporous membrane.

11. The system of claim 7, wherein the nanoporous membrane is comprised of at least one material selected from the group consisting of polyester, polyimide, polycarbonate, carbon nanotubes, silicon, silica, alumina, and ceramic.

12. The system of claim 1, wherein the gate region comprises a hydrogel polymer.

13. The system of claim 12, wherein the hydrogel polymer is neutral.

14. The system of claim 12, wherein the hydrogel polymer is charged.

15. The system of claim 1, wherein the gate region comprises a plurality of gate channels providing fluidic communication between the drain and source regions and wherein a width of a respective gate channel is less than one tenth a width of the interface of the respective gate channel and the drain or source region.

16. A method for manipulating charged analytes, comprising:
providing a device having a drain region, a source region, and a gate region disposed therebetween;
associating a first electrolyte solution with the drain region and a second electrolyte solution with the source region, wherein the first or second electrolyte solution contains a charged analyte; and
producing a first electric field between the drain and source regions such that the charged analyte moves under the electric field towards the gate region, and an electroosmotic flow-induced convective flow moves through the gate region in a direction opposite to the motion of the charged analyte under the electric field, whereby the charged analyte becomes concentrated due to the opposing motions.

17. The method of claim 16, wherein the charged analyte is a biomolecule.

18. The method of claim 16, wherein the charged analyte is DNA or RNA.

19. The method of claim 16, wherein the charged analyte is a cell.

20. The method of claim 16, wherein the charged analyte is a bead particle.

21. The method of claim 16, wherein the nanoporous membrane is a polyester membrane.

22. The system of claim 16, further comprising detecting motion of the charged analyte.

23. The system of claim 16, further comprising labeling the charged analyte with a label.

24. The method of claim 16, further comprising reversing the bias caused by the first electric field.

25. The method of claim 16, further comprising:
providing a separation region coupled to the source region near a point of intersection between the gate region and the source region; and
after producing the first electric field, producing a second electric field within the separation channel such that components of the charged analyte are substantially separated along the separation region.

26. A method for manipulating charged analytes, comprising:
providing a device having a drain region, a source region, a separation region, and a gate region disposed between the drain region and the source region;
associating a buffer solution with the drain region, the source region, and the separation region;
causing a potential difference between the drain region and the source region until a stable current is reached;
replacing the buffer solution in the source region with a solution containing an analyte;
applying a negative potential to the source region and a positive potential to the drain region to create a forward bias between the source region and the drain region;
removing the forward bias between the source region and the drain region; and
applying a bias between the source region and the separation region.

27. The system of claim 26, further comprising detecting motion of the analyte.

28. The system of claim 26, further comprising labeling the analyte with a label.

29. The system of claim 26, wherein the bias between the source region and the separation region is a forward bias operable to direct an enriched band of analytes toward the separation region.

30. The system of claim 25, wherein the bias between the source region and the separation region is a reverse bias operable to direct an enriched band of analytes toward the separation region.

31. A method for manipulating charged analytes, comprising:
providing a device having a first drain region, a second drain region, a source region, a first gate region disposed between the first drain region and the source region, and a second gate region disposed between the second drain region and the source region;
associating a buffer solution with the first drain region, the second drain region, and the source region;
causing a potential difference between the first drain region and the source region until a stable current is reached;
replacing the buffer solution in the source region with a solution containing an analyte;
applying a first negative potential to the source region to create a forward bias between the source region and the first drain region, thereby creating an enriched band of the analyte adjacent the first gate region;
removing the first negative potential; and
applying a second negative potential to the source region to create a forward bias between the source region and the second drain region, thereby moving the enriched band of the analyte toward the second gate region.

32. The system of claim 31, further comprising detecting motion of the analyte.

33. The system of claim 31, further comprising detecting motion of the enriched band of the analyte.

34. The system of claim 31, further comprising labeling the analyte with a label.

35. A method for manipulating charged analytes, comprising:
providing a device having a drain region, a source region, and a series of gate channels having successively smaller cross-sectional areas disposed therebetween;
associating a buffer solution with the drain region and the source region;
causing a potential difference between the drain region and the source region until a stable current is reached;
replacing the buffer solution in the source region with a solution containing a plurality of analytes; and
applying a negative potential to the source region to create a forward bias.

36. The system of claim 35, further comprising detecting motion of the analytes.

37. The system of claim 35, further comprising labeling at least one of the analytes with a label.

38. A method for concentrating charged analytes within a microfluidic device, comprising:
providing a microfluidic device having a unit comprising a first channel section and an associated first electrode, a second channel section and an associated second electrode, and a hydrogel plug separating the first and second channel sections while providing fluidic communication therebetween;
associating a first electrolyte solution with the first channel section and a second electrolyte solution with the second channel section, wherein either the first or second electrolyte solution comprises a charged analyte;
producing an electric field between the first and second electrodes such that the charged analyte moves under the electric field towards the hydrogel, and an electroosmotic flow-induced convective flow moves through the hydrogel in a direction opposite to the motion of the charged analyte under the electric field, whereby the charged analyte becomes concentrated due to the opposing motions.

39. The method of claim 38, wherein the hydrogel is neutral.

40. The method of claim 38, wherein the hydrogel is charged.

41. The method of claim 40, wherein the charge is negative.

42. The method of claim 38, wherein the hydrogel is comprised of acrylate.

43. The method of claim 38, wherein the hydrogel is a crosslinked hydrogel polymer.

44. The method of claim 43, wherein the crosslinked hydrogel is prepared using ethylene glycol dimethacrylate.

45. The method of claim 43, wherein the crosslinked hydrogel is prepared using acrylic acid.

46. A method for manipulating charged analytes, comprising:
providing a device having a drain region, a source region, and a nanoporous membrane separating the drain region and the source region;
associating a first solution with the drain region
associating a second solution containing an analyte with the source region;
causing an electrophoretic velocity of the analyte in the second solution to be greater than an electroosmotic velocity of the first solution; and
causing a local velocity of the first solution exiting the pores of the membrane to be greater than the electrophoretic velocity of the analyte in the second solution.
